(19) United States
(12) Patent Application Publication
(10) Pub. No.: US 2006/0233843 A1
(43) Pub. Date: Oct. 19, 2006

(54) Treatment of psychosis with a muscarinic m1 receptor ectopic activator

(76) Inventors: P. Jeffrey Conn, Brentwood, TN (US); Marlene A. Jacobson, Melrose Park, PA (US); Pierre J. Mallorga, Lansdale, PA (US); Edward M. Scolnick, Wynniwood, PA (US); Cyrille Sur, Harleysville, PA (US)

Correspondence Address: MERCK AND CO., INC, P O BOX 2000, RAHWAY, NJ, 07065-0907, US

(21) Appl. No.: 10/545,767

(22) PCT Filed: Feb. 17, 2004
(86) PCT No.: PCT/US04/04735

§ 371 Date: May 10, 2006

Related U.S. Application Data
(60) Provisional application No. 60/448,365, filed on Feb. 19, 2003.

(51) Int. Cl. A61K 31/5415; A61K 31/4439; A61K 31/4745; A61K 9/00

(52) U.S. Cl. 424/400; 514/225.8; 514/283

(57) ABSTRACT

A muscarinic M1 receptor ectopic activator, such as a muscarinic M1 receptor allosteric potentiator or a muscarinic M1 receptor ectopic agonist is useful, alone or in combination with other antipsychotic agents, for treating or preventing psychosis, such as a schizophrenic disorder or psychosis in Alzheimer's disease or bipolar disorder, for enhancing cognition and for neuropathic pain.

### BACKGROUND OF THE INVENTION

Schizophrenia is among the most debilitating psychiatric disorders and has a considerable social and economic impact as it affects approximately 1% of the world's population. For instance, schizophrenia is more prevalent than Alzheimer's disease, multiple sclerosis and even diabetes. In the United States, around 2.5 million persons are affected with a cost of $40 billion/year if productivity losses are included. The essential features of schizophrenia are a mixture of characteristic signs and symptoms (both positive and negative) which are present in an individual for a significant portion of time over at least one month. The so-called “active-phase” symptoms include delusions, hallucinations, disorganized speech, disorganized or catatonic behavior and negative symptoms (e.g. affective flattening, alogia and avolition). Some patients have only a single episode of the illness, but most have either recurrent episodes or chronic illness.

The care of schizophrenic patients is a major part of the work of psychiatrists. The long-term care of schizophrenic patients is complicated. Generally, symptoms can at least be kept under control if patients with chronic schizophrenia receive long-term treatment with an antipsychotic drug. Frequently, schizophrenic symptoms cannot be controlled without invoking extrapyramidal side-effects. Consequently, antiparkinsonian drugs may also be prescribed to reduce these side-effects. However, the use of anticholinergic drugs may actually increase the risk of tardive dyskinesia (a late and sometimes irreversible side-effect of prolonged treatment with antipyschotic drugs).

Numerous compounds are disclosed in the art for treating or preventing psychosis, such as a schizophrenic disorder, including e.g., sedatives, hypnotics, typical antipsychotics, atypical antipsychotics, and the like. There are a number of major drawbacks with currently used typical and atypical antipsychotic treatments. These include significant side effect liability as well as lack of complete efficacy in ameliorating psychotic symtoms. The typical antipsychotics possess antipsychotic and sedative properties to varying degrees and are generally effective against positive symptoms, but are not generally effective against negative symptoms and may even exacerbate them. Typical antipsychotic drugs also have a propensity to induce disabling and ultimately disfiguring Parkinson-like extrapyramidal motor symptoms, such as tardive dyskinesia. Treatment of schizophrenia with antipsychotic (or neuroleptic) agents, such as haloperidol and chlorpromazine, is typically associated with a number of side-effects, including extrapyramidal symptoms, acute dystonias, tardive dyskinesias, akathesia, tremor, tachycardia, drowsiness, confusion, postural hypotension, blurring of vision, precipitation of glaucoma, dry mouth, constipation, urinary hesitance and impaired sexual function. Such side-effects are often debilitating and contribute considerably to a patient's non-compliance with prescribed treatment. They may also hinder a patient's social rehabilitation. The atypical antipsychotics offer modest efficacy against negative symptoms and relatively improved tolerability with respect to extrapyramidal motor symptoms, and are all associated with specific adverse events. In view of the short-comings of existing antipsychotic therapy, and the inability of the currently prevailing dopamine hyperfunction hypothesis to fully account for the pathophysiology of schizophrenia, there is a need for new, safe and effective treatment for schizophrenic disorders.

### SUMMARY OF THE INVENTION

The present invention is directed to the use of a compound which has the ability to ectopically activate the G-protein coupled muscarinic acetylcholine M1 receptor, such as a muscarinic M1 receptor allosteric potentiator or a muscarinic M1 receptor ectopic agonist, alone or in combination with other antipsychotic agents, for treating or preventing psychosis, such as a schizophrenic disorder or psychosis in Alzheimer's disease or bipolar disorder, for enhancing cognition and for neuropathic pain. The present invention further provides a pharmaceutical composition for treating or preventing psychosis, such as a schizophrenic disorder or psychosis in Alzheimer's disease or bipolar disorder, for enhancing cognition and for neuropathic pain.

### DESCRIPTION OF THE INVENTION

The present invention is directed to the use of a compound which has the ability to ectopically activate the G-protein coupled muscarinic acetylcholine M1 receptor, such as a muscarinic M1 receptor allosteric potentiator or a muscarinic M1 receptor ectopic agonist, alone or in combination with other antipsychotic agents, for treating or preventing psychosis, such as a schizophrenic disorder or psychosis in Alzheimer's disease or bipolar disorder, for enhancing cognition and for neuropathic pain. The present invention further provides a pharmaceutical composition for treating or preventing psychosis, such as a schizophrenic disorder or psychosis in Alzheimer's disease or bipolar disorder, for enhancing cognition and for neuropathic pain. Another embodiment of the present invention is directed to a method for the treatment, control, amelioration or reduction of risk of a disease or disorder where abnormal oscillatory activity occurs in the brain, including depression, migraine, neuropathic pain, Parkinson's disease, psychosis and schizophrenia, as well as diseases or disorders where there is abnormal coupling of activity, particularly through the thalamus.

By the term “muscarinic M1 receptor ectopic activator” is meant any exogenously administered compound or agent that directly or indirectly augments the activity of the muscarinic M1 receptor, in the presence or in the absence of the endogenous ligand (such as acetylcholine) for the muscarinic M1 receptor, in an animal, in particular, a human, but does not interact with the orthosteric site of the muscarinic M1 receptor.

By the term “muscarinic M1 receptor allosteric potentiator” is meant any exogenously administered compound or agent that directly or indirectly augments the response produced by the endogenous ligand (such as acetylcholine) at the orthosteric site of the muscarinic M1 receptor in an animal, in particular, a human. Because it does not induce desensitization of the receptor, the use of a muscarinic M1 receptor allosteric potentiator would provide unexpected advantages over the use of a muscarinic M1 receptor ectopic agonist. Such advantages may include, for example, increased safety margin, higher tolerability, diminished potential for abuse, and reduced toxicity.

By the term “muscarinic M1 receptor ectopic agonist” is meant any exogenously administered compound or agent that indirectly augments the activity of the muscarinic M1 receptor in an animal, in particular, a human. The muscarinic M1 receptor ectopic agonist binds to a site on the muscarinic M1 receptor that is distinct from the orthosteric acetylcholine site of the muscarinic M1 receptor. In contrast to an “allosteric agonist” which directly influences the orthosteric site of the muscarinic M1 receptor, the ectopic agonist may indirectly or directly influence the orthosteric site of the muscarinic M1 receptor.

In an embodiment of the present invention the muscarinic M1 receptor ectopic activator possesses a selectivity for the muscarinic M1 receptor relative to each of the muscarinic M2, M3 and MS receptors of at least 5 fold as measured by the ratio of EC₅₀ for the muscarinic M1 receptor to the EC₅₀ for each of the muscarinic M2, M3 and M5 receptors as evaluated by the Muscarinic FLIPR assay.

In another embodiment of the present invention the muscarinic M1 receptor ectopic activator possesses a selectivity for the muscarinic M1 receptor relative to each of the muscarinic M2, M3 and M5 receptors of at least 10 fold as measured by the ratio of EC50 for the muscarinic M1 receptor to the EC₅₀ for each of the muscarinic M2, M3 and M5 receptors as evaluated by the Muscarinic FLIPR assay.

In another embodiment of the present invention the muscarinic M1 receptor ectopic activator possesses a selectivity for the muscarinic M1 receptor relative to each of the muscarinic M2, M3 and M5 receptors of at least 50 fold as measured by the ratio of EC₅₀ for the muscarinic M1 receptor to the EC₅₀ for each of the muscarinic M2, M3 and M5 receptors as evaluated by the Muscarinic FLIPR assay.

In another embodiment of the present invention the muscarinic M1 receptor ectopic activator possesses a selectivity for the muscarinic M1 receptor relative to each of the muscarinic M2, M3 and M5 receptors of at least 100 fold as measured by the ratio of EC₅₀ for the muscarinic M1 receptor to the EC₅₀ for each of the muscarinic M2, M3 and M5 receptors as evaluated by the Muscarinic FLIPR assay.

In another embodiment of the present invention the muscarinic M1 receptor ectopic activator possesses a selectivity for the muscarinic M1 receptor relative to each of the muscarinic M2, M3 and M5 receptors of at least 200 fold as measured by the ratio of EC₅₀ for the muscarinic M1 receptor to the EC₅₀ for each of the muscarinic M2, M3 and M5 receptors as evaluated by the Muscarinic FLIPR assay.

In another embodiment of the present invention the muscarinic M1 receptor ectopic activator possesses a selectivity for the muscarinic M1 receptor relative to each of the muscarinic M2, M3 and M5 receptors of at least 500 fold as measured by the ratio of EC₅₀ for the muscarinic M1 receptor to the EC₅₀ for each of the muscarinic M2, M3 and M5 receptors as evaluated by the Muscarinic FLIPR assay.

In an embodiment of the present invention the muscarinic M1 receptor ectopic activator possesses a selectivity for the muscarinic M1 receptor relative to the muscarinic M4 receptor of at least 5 fold as measured by the ratio of EC₅₀ for the muscarinic M1 receptor to the EC₅₀ of the muscarinic M4 receptor as evaluated by the Muscarinic FLIPR assay.

In another embodiment of the present invention the muscarinic M1 receptor ectopic activator possesses a selectivity for the muscarinic M1 receptor relative to the muscarinic M4 receptor of at least 50 fold as measured by the ratio of EC₅₀ for the muscarinic M1 receptor to the EC₅₀ of the muscarinic M4 receptor as evaluated by the Muscarinic FLIPR assay.

In another embodiment of the present invention the muscarinic M1 receptor ectopic activator possesses a selectivity for the muscarinic M1 receptor relative to the muscarinic M4 receptor of at least 100 fold as measured by the ratio of EC₅₀ for the muscarinic M1 receptor to the EC₅₀ of the muscarinic M4 receptor as evaluated by the Muscarinic FLIPR assay.

In another embodiment of the present invention the muscarinic M1 receptor ectopic activator possesses a selectivity for the muscarinic M1 receptor relative to the muscarinic M4 receptor of at least 200 fold as measured by the ratio of EC₅₀ for the muscarinic M1 receptor to the EC₅₀ of the muscarinic M4 receptor as evaluated by the Muscarinic FLIPR assay.

In an embodiment of the present invention the muscarinic M1 receptor ectopic activator possesses a selectivity for the muscarinic M1 receptor relative to the dopamine D2 receptor of at least 5 fold as measured by the ratio of EC₅₀ for the muscarinic M1 receptor as evaluated by the Muscarinic FLIPR assay to the EC₅₀ of the dopamine D2 receptor.

In another embodiment of the present invention the muscarinic M1 receptor ectopic activator possesses a selectivity for the muscarinic M1 receptor relative to the dopamine D2 receptor of at least 50 fold as measured by the ratio of EC₅₀ for the muscarinic M1 receptor as evaluated by the Muscarinic FLIPR assay to the EC₅₀ of the dopamine D2 receptor.

In another embodiment of the present invention the muscarinic M1 receptor ectopic activator possesses a selectivity for the muscarinic M1 receptor relative to the dopamine D2 receptor of at least 100 fold as measured by the ratio of EC₅₀ for the muscarinic M1 receptor as evaluated by the Muscarinic FLIPR assay to the EC₅₀ of the dopamine D2 receptor.

In another embodiment of the present invention the muscarinic M1 receptor ectopic activator possesses a selectivity for the muscarinic M1 receptor relative to the dopamine D2 receptor of at least 200 fold as measured by the ratio of EC₅₀ for the muscarinic M1 receptor as evaluated by the Muscarinic FLIPR assay to the EC₅₀ of the dopamine D2 receptor.

In an embodiment of the present invention the muscarinic M1 receptor ectopic activator possesses a selectivity for the muscarinic M1 receptor relative to all 5HT receptors of at least 5 fold as measured by the ratio of EC₅₀ for the muscarinic M1 receptor as evaluated by the Muscarinic FLIPR assay to the EC₅₀ of each of the 5HT receptors.

In another embodiment of the present invention the muscarinic M1 receptor ectopic activator possesses a selectivity for the muscarinic M1 receptor relative to all 5HT receptors of at least 50 fold as measured by the ratio of EC₅₀ for the muscarinic M1 receptor as evaluated by the Muscarinic FLIPR assay to the EC₅₀ of each of the 5HT receptors.

In another embodiment of the present invention the muscarinic M1 receptor ectopic activator possesses a selectivity for the muscarinic M1 receptor relative to all 5HT receptors of at least 100 fold as measured by the ratio of EC₅₀ for the muscarinic M1 receptor as evaluated by the Muscarinic FLIPR assay to the EC₅₀ of each of the 5HT receptors.

In another embodiment of the present invention the muscarinic M1 receptor ectopic activator possesses a selectivity for the muscarinic M1 receptor relative to all 5HT receptors of at least 200 fold as measured by the ratio of EC₅₀ for the muscarinic M1 receptor as evaluated by the Muscarinic FLIPR assay to the EC₅₀ of each of the 5HT receptors.

In an embodiment of the present invention the muscarinic M1 receptor ectopic activator acts at a different site than the orthosteric site of the muscarinic M1 receptor. In an embodiment of the present invention the muscarinic M1 receptor ectopic activator acts at a different site from the ectopic site for the compound AC42, which is disclosed in WO 99/50247 as an agonist for an M1 receptor ectopic site. The agonist activity of AC-42 is mediated in part by residues 1-45 and 388-418 of the M1 receptor. See Spalding et al., Mol Pharmacol, 61:1297-1302 (2002). Mutation of residue 381 from tyrosine to alanine increased the ectopic agonist potency of N-desmethylclozapine by 8-fold, while not affecting the pharmacology of AC-42. See Sur et al., PNAS, 100:13674-13679 (2003). It is believed that residue 381, located within the orthosteric site, is not critical for the activation of the muscarinic M1 receptor by the ectopic activator of the invention.

In an embodiment of the present invention the muscarinic M1 receptor ectopic activator possesses an EC₅₀ for binding to the muscarinic M1 receptor of 1 μM or less as evaluated by the Muscarinic FLIPR assay.

In another embodiment of the present invention the muscarinic M1 receptor ectopic activator possesses an EC₅₀ for binding to the muscarinic M1 receptor of 500 nM or less as evaluated by the Muscarinic FLIPR assay.

In another embodiment of the present invention the muscarinic M1 receptor ectopic activator possesses an EC₅₀ for binding to the muscarinic M1 receptor of 100 nM or less as evaluated by the Muscarinic FLIPR assay.

In another embodiment of the present invention the muscarinic M1 receptor ectopic activator possesses an EC₅₀ for binding to the muscarinic M1 receptor of 50 nM or less as evaluated by the Muscarinic FLIPR assay.

In another embodiment of the present invention the muscarinic M1 receptor ectopic activator possesses an EC₅₀ for binding to the muscarinic M1 receptor of 1 nM or less as evaluated by the Muscarinic FLIPR assay.

In an embodiment of the present invention the muscarinic M1 receptor ectopic activator is an orally active muscarinic M1 receptor ectopic activator. In an embodiment of the present invention the muscarinic M1 receptor ectopic activator is orally administered. In another embodiment of the present invention the muscarinic M1 receptor ectopic activator is a non-peptidal muscarinic M1 receptor ectopic activator.

In a specific embodiment of the present invention, the muscarinic M1 receptor ectopic activator is desmethylclozapine which is a muscarinic M1 receptor ectopic agonist.
Desmethylclozapine has the structure: 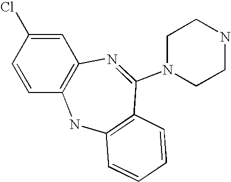
and may be prepared by methods well known in the art (e.g., Eur. J. Pharmacol., 245(2):179-92 (1996)).

In a specific embodiment of the present invention, the muscarinic M1 receptor ectopic activator is brucine, which is a muscarinic M1 receptor allosteric potentiator. Brucine may be named as 2,3-dimethoxystrychnidin-10-one and has the structure: 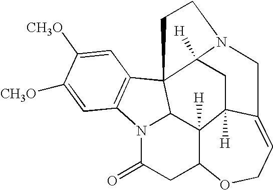
and may be prepared by methods well known in the art (e.g., E. Tedeschi et al, Tetrahedron, 24, 4573 (1968)).

The muscarinic M1 receptor ectopic activator may be peptidal or non-peptidal in nature, however, the use of a non-peptidal muscarinic M1 receptor ectopic activator is preferred. In addition, for convenience the use of an orally active muscarinic M1 receptor ectopic activator is preferred. Similarly, for convenience the use of a once-a-day medicament is preferred.

In an embodiment of the present invention the muscarinic M1 receptor ectopic activator is a CNS-penetrant muscarinic M1 receptor ectopic activator and is able to enter the brain and/or central nervous system with sufficient concentration to have a therapeutic effect. In a further embodiment of the present invention the CNS-penetrant muscarinic M1 receptor ectopic activator is a compound that exhibits sufficient concentration in the brain and/or central nervous system to have therapeutic efficacy upon oral administration.

The exceptional pharmacology of the muscarinic M1 receptor ectopic activators of use in the present invention enables the treatment of psychosis and the other subject indications, without the need for concomitant therapy and in particular, without the need for concomitant use of antipsychotic agents.

In an embodiment of the present invention the muscarinic M1 receptor ectopic activator has an onset of action of 45-60 minutes.

In an embodiment of the present invention the muscarinic M1 receptor ectopic activator has a pharmacological half life (T½ life) of short duration. In another embodiment of the present invention the muscarinic M1 receptor ectopic activator has a pharmacological half life (T½ life) of intermediate duration. In another embodiment of the present invention the muscarinic M1 receptor ectopic activator has a pharmacological half life (T½ life) of long duration. In another embodiment of the present invention the muscarinic M1 receptor ectopic activator has a pharmacological half life (T½ life) of at least about 2 hours duration.

The muscarinic M1 receptor ectopic activator may be used alone or in combination with other muscarinic M1 receptor ectopic activators or with other agents which are known to be beneficial in the subject indications. The muscarinic M1 receptor ectopic activator and the other agent may be co-administered, either in concomitant therapy or in a fixed combination. For example, the muscarinic M1 receptor ectopic activator may be administered in conjunction with other compounds which are known in the art for the subject indications, including e.g., sedatives, hypnotics, anxiolytics, antipsychotics, antianxiety agents, cyclopyrrolones, imidazopyridines, pyrazolopyrimidines, minor tranquilizers, melatonin agonists and antagonists, melatonergic agents, benzodiazepines, barbiturates, 5HT-2 antagonists, and the like, such as: adinazolam, allobarbital, alonimid, alprazolam, amitriptyline, amobarbital, amoxapine, bentazepam, benzoctamine, brotizolam, bupropion, buspirone, butabarbital, butalbital, capuride, carbocloral, chloral betaine, chloral hydrate, chlordiazepoxide, clomipramine, clonazepam, cloperidone, clorazepate, clorethate, clozapine, cyprazepam, desipramine, dexclamol, diazepam, dichloralphenazone, divalproex, diphenhydramine, doxepin, estazolam, ethchlorvynol, etomidate, fenobam, flunitrazepam, flurazepam, fluvoxamine, fluoxetine, fosazepam, glutethimide, halazepam, hydroxyzine, imipramine, lithium, lorazepam, lormetazepam, maprotiline, mecloqualone, melatonin, mephobarbital, meprobamate, methaqualone, midaflur, midazolam, nefazodone, nisobamate, nitrazepam, nortriptyline, oxazepam, paraldehyde, paroxetine, pentobarbital, perlapine, perphenazine, phenelzine, phenobarbital, prazepam, promethazine, propofol, protriptyline, quazepam, reclazepam, roletamide, secobarbital, sertraline, suproclone, temazepam, thioridazine, tracazolate, tranylcypromaine, trazodone, triazolam, trepipam, tricetamide, triclofos, trifluoperazine, trimetozine, trimipramine, uldazepam, venlafaxine, zaleplon, zolazepam, zolpidem, and salts thereof, and combinations thereof, and the like, or the muscarinic M1 receptor ectopic activator may be administered in conjunction with the use of physical methods such as with light therapy or electrical stimulation.

In the above structural formulas and throughout the instant specification, the following terms have the indicated meanings.

Certain of the above defined terms may occur more than once in the above formula and upon such occurrence each term shall be defined independently of the other. Similarly, the use of a particular variable within a noted structural formula is intended to be independent of the use of such variable within a different structural formula.

For use in medicine, the salts of the compounds employed in this invention refer to non-toxic “pharmaceutically acceptable salts.” Other salts may, however, be useful in the preparation of the compounds according to the invention or of their pharmaceutically acceptable salts. Salts encompassed within the term “pharmaceutically acceptable salts” refer to non-toxic salts of the compounds of this invention which are generally prepared by reacting the free base with a suitable organic or inorganic acid. Representative salts include the following: Acetate, Benzenesulfonate, Benzoate, Bicarbonate, Bisulfate, Bitartrate, Borate, Bromide, Calcium, Camsylate, Carbonate, Chloride, Clavulanate, Citrate, Dihydrochloride, Edetate, Edisylate, Estolate, Esylate, Fumarate, Gluceptate, Gluconate, Glutamate, Glycollylarsanilate, Hexylresorcinate, Hydrabamine, Hydrobromide, Hydrochloride, Hydroxynaphthoate, Iodide, Isothionate, Lactate, Lactobionate, Laurate, Malate, Maleate, Mandelate, Mesylate, Methylbromide, Methylnitrate, Methylsulfate, Mucate, Napsylate, Nitrate, N-methylglucamine ammonium salt, Oleate, Oxalate, Pamoate (Embonate), Palmitate, Pantothenate, Phosphate/diphosphate, Polygalacturonate, Salicylate, Stearate, Subacetate, Succinate, Sulfate, Sulfonate, Tannate, Tartrate, Teoclate, Tosylate, Triethiodide and Valerate. Furthermore, where the compounds of the invention carry an acidic moiety, suitable pharmaceutically acceptable salts thereof may include alkali metal salts, e.g., sodium or potassium salts; alkaline earth metal salts, e.g., calcium or magnesium salts; and salts formed with suitable organic ligands, e.g., quaternary ammonium salts.

The compounds employed in the present invention, may have chiral centers and occur as racemates, racemic mixtures and as individual diastereomers, or enantiomers with all isomeric forms being included in the present invention. Therefore, where a compound is chiral, the separate enantiomers, substantially free of the other, are included within the scope of the invention; further included are all mixtures of the two enantiomers.

Members of the G-protein coupled muscarinic acetylcholine receptor family denoted as the muscarinic M1, M2, M3, M4 and M5 receptor subtypes are fully disclosed in e.g., Pharmacol. Ther., 58:319-379 (1993); Eur. J. Pharmacol., 295:93-102 (1996); Mol. Pharmacol., 61:1297-1302 (2002); PCT WO 01/29036; PCT WO 01/83472.

The identification of a compound as a muscarinic M1 receptor ectopic activator may be readily determined without undue experimentation by methodology well known in the art, including the “Muscarinic FLIPR Assay”. A typical assay is conducted essentially as follows.

Muscarinic FLIPR Assay

This assay is designed to select compounds that possess modulator activity at the acetylcholine muscarinic M1 receptor or mutants thereof (or other muscarinic receptors) expressed in CHOK1 or CHOnfat cells by measuring the intracellular Calcium with a FLIPR³⁸⁴ Fluorometric Imaging Plate Reader System.

This assay determines the modulator capacity of compounds when administered to cells transfected with the human muscarinic M1 receptor. The assay studies the effect of one or several concentrations of test compounds on basal or acetylcholine-stimulated Ca²⁺ levels using FLIPR. (All compounds are prepared at a working concentration of 3×.) After a preincubation period of 4 minutes with these test compounds, a single EC₂₀ concentration of acetylcholine is added to each well (1 nM final). The intracellular Ca²⁺ level of each sample is measured and compared to an acetylcholine control to determine any modulatory activity.

Cells: CHOK1/hM1 CHOnfat/hM1 cells are plated 24 hours before the assay at a density of 15,000 cells/well (100 μL) in a 384 well plate, (Poly-D-Lysine Black/Clear Microtest (TM) Tissue-Culture Treated Polystyrene, Becton Dickinson #35 6663). CHOK1 Growth Medium (for 500 ml): 85% DMEM (Hi Glucose) GIBCO Cat. #11965-084) 425 ml; 10% H FBS (GIBCO Cat #16140-063) 50 ml; 2 mM L-glutamine (GIBCO Cat. #25030-149) 5 ml (200 mM stock); 0.1 mM NEAA (GIBCO Cat. #11140-050) 5 ml (10 mM stock); Pen-Strep (GIBCO Cat. #15140-148) 5 ml stock; 1 mg/ml Geneticin (GIBCO Cat#10131-019) 10 ml (50 mg/ml stock), are added.

Equipment: 384 well plate, 120 μL addition plate (Becton Dickinson #356663); 96-well Whatman 2 ml Uniplate (Whatman Cat. No. 7701-5200) Incubator, 37° C., 5% CO₂; Skatron EMBLA-384 Plate Washer; Multimek Pipetting System; FLIPR³⁸⁴ Fluorometric Imaging Plate Reader System.

Buffers. Assay Buffer: Hanks Balanced Salt Solution (GIBCO Cat. #14025-076), with 20 mM Hepes (GIBCO Cat. #15630-080), 2.5 mM Probenecid (Sigma P-8761) first dissolved in 1N NaOH, 1% Bovine Serum Albumin (Sigma A-9647). Dye Loading Buffer: Assay Buffer (see above) plus 1% Fetal Bovine Serum (GIBCO Cat #16140-063) and Fluo-4AM/Pluronic Acid Mixture—(First mix together before adding to Dye Loading Buffer). 2 mM Fluo-4AM ester stock in DMSO (Molecular Probes F-14202) Concentration of 2 uM in buffer for a Final concentration of 1 uM in Assay. 20% Pluronic Acid Solution stock (Molecular Probes P-3000) Concentration of 0.04% In Buffer, 0.02% in Assay.

Example for loading four 384 well plates: Mix 65 uL of 2 mM Fluo-4AM with 130 uL of 20% Pluronic Acid. Add resulting solution and 650 uL FBS to Assay Buffer for a total volume of 65 mL. Positive Controls: 4-Br-A23187 (Molecular Probes Cat #B-1494): 10 mM in DMSO; final concentration 10 μM. Acetylcholine (Sigma A-6625): 10 mM in water, working stock at both 2 mM and 3 mM in assay buffer, final concentration is 1 mM. This is used to check the maximum stimulation of the CHOK1/hM1 cells. 2 mM (2×) Acetylcholine is added in the preincubation part of the assay, and the 3 mM (3×) stock is added in the second part. Brucine, sulfate hydrate, 99% (Sigma-Aldrich #237868) 10 mM in water, 300 μM working stock in 3% DMSO (3×), 100 μM final concentration. Preincubation with Brucine serves as the modulator positive control when acting with EC₂₀ Acetylcholine. (EC₂₀)Acetylcholine: 10 mM in water, working stock of 3 nM (3×), final concentration in assay is 1 nM. This is used after the preincubation with test compounds. Addition of the EC₂₀ Acetylcholine to each well with a test compound will ascertain any modulator activity. 8 wells contain 1 nM Acetylcholine alone as a control.

Determining Activity of Putative Compounds: Compounds are applied to a 96-well plate, 100% DMSO, at a concentration of 2 mM. They are diluted in assay buffer to 60 μM (3× working concentration) in a Whatman 2 ml Uniplate, columns 2-11. The final concentration in assay is 20 μM, 1% DMSO. Assay Setup.

Screening Plate: In a 96-well Whatman 2 ml Uniplate containing the 3× screening compounds, transfer the 3× Brucine control to wells B1 and C1. Pipet the 3 mM Acetylcholine control (3×) into well H1, and assay buffer into remaining wells (Basal is Column 12). Using Multimek, transfer the 3× plate into a 384 well plate, which allows quadruplicate data points for each compound in assay. Agonist Plate: In a separate 96-well Whatman 2 ml Uniplate, pipet 3 nM Acetylcholine (3×) into wells corresponding to the screening compounds (columns 2-11), and into wells B1, C1, D1, and E1. Pipet the 3 mM Acetylcholine control (3×) into wells F1 and G1, and assay buffer into the remaining wells (Basal is Column 12). Using Multimek, transfer the 3× agonist plate into a 384 well plate.

Cell Washing and Dye Loading: Cells are washed three times with 100 μL of buffer with the Skatron EMBLA 384 Plate washer (Program 1 on EMBLA Cell Wash, WP46-3010 FLIPR lab). This program leaves 30 μL of buffer in each well. Using Multimek, pipet 30 μL of Dye Loading Buffer into each well. Incubate at 37° C., 5% CO₂ for up to one hour.

FLIPR Assay: After 60 minutes, the cells are washed three times with 100 μL of buffer with the Skatron EMBLA 384 Plate washer (Program 1 on EMBLA Cell Wash, WP46-3010 FLIPR lab); 30 μL of buffer is left in each well. Place cell plate, screening plate, and agonist addition plates on the platform in the FLIPR and close door. Perform signal test to check background fluorescence and basal fluorescence signal. Laser intensity is adjusted if necessary.

Modulator Assay: Provide 4 minutes of preincubation with the test compounds to determine any agonist activity on the M1 receptor by comparison to the 1 mM Acetylcholine control. After preincubation, the EC₂₀ value of acetylcholine (1 nM final) is added to determine any modulator activity. Data files are prepared in Excel. The first file contains the maximum counts from the modulator part of assay, and the second file contains the maximum counts from the preincubation. Preincubation (Agonist) Data: Calculate the average for the basal counts, and subtract this value from all data points. Next, average the counts for each set of quadruplicate points for the screening compounds (A) and for the 1 mM Acetylcholine control (B). Divide the average compound (A) by average acetylcholine (B) and multiply by 100% to calculate the percent of maximum stimulation for the compound (C). (A/B)×100%=C (Percent of Maximum Stimulation). Modulator Data: Calculate the average for the basal counts, and subtract this value from all data points. Next, average the counts for each set of quadruplicate points for the compound(s) in the presence of 1 nM acetylcholine (D) and for the 1 nM acetylcholine control (E). Divide first value (D) by the second value (E) to determine the fold stimulation (F). The activity of brucine (100 μM) as a potentiator is also calculated using the same formula. D/E=F (Fold Stimulation of 1 nM acetylcholine)

Agonist Assay: This assay is used for determining the agonist potency of certain compounds on CHOK1/hM1 cells. For agonist addition plate, refer to agonist plate as described above, except prepare agonist 2× concentration as opposed to 3× for modulator assay. Include positive control 20 μM A-23187 (2×, 10 μM final). Data files are prepared in Excel. Calculate the average for the basal counts, and subtract this value from all data points (G). If several concentrations are used, calculate EC₅₀ and the maximum stimulation (Emax) for this compound by plotting G vs concentration in a dose response curve in Prism, Excel, or Sigma Plot. Calculate percent of maximum stimulation (I) by dividing average agonist counts (G) or Emax by average 1 mM acetylcholine (H) counts multiplied by 100%. (G/H)×100%=I (Percent of Maximum Stimulation)

Antagonist Assay: This assay is used for determining the antagonist activity of certain compounds on CHOK1/hM1 cells. All compounds are prepared 3× final concentration. Cells are preincubated with compound of interest for 4 minutes, followed by the addition of a single EC₈₀ point (3 nM) of acetylcholine. Data files are prepared in Excel. Calculate the average for the basal counts, and subtract this value from all data points(G). If several concentrations of antagonist are used, calculate IC₅₀ by plotting G vs concentration in a dose response curve in Prism, Excel, or Sigma Plot. When one concentration is used calculate percent inhibition (L) by dividing average G by average 3 nM acetylcholine counts (K); multiply by 100%. Subtract this number from 100. (100−(J/K))×100%=L (Percent Inhibition).

The intrinsic activity of the muscarinic M1 receptor ectopic activator compounds which may be used in the present invention may be determined by these assays.

In accordance with the present invention, the muscarinic M1 receptor ectopic activator, such as a muscarinic M1 receptor allosteric potentiator or a muscarinic M1 receptor ectopic agonist, is useful alone or in combination with other antipsychotic agents for treating, controlling, ameliorating or reducing the risk of psychosis, a schizophrenic disorder, psychosis in Alzheimer's disease, psychosis in bipolar disorder, for enhancing cognition and for treating, controlling, ameliorating or reducing the risk of neuropathic pain. As used herein, the term “schizophrenic disorder” includes paranoid, disorganized, catatonic, undifferentiated and residual schizophrenia; schizophreniform disorder; schizoaffective disorder; delusional disorder; brief psychotic disorder; shared psychotic disorder; substance-induced psychotic disorder; and psychotic disorder not otherwise specified. Other conditions commonly associated with schizophrenic disorders include self-injurious behaviour (e.g. Lesch-Nyhan syndrome) and suicidal gestures.

The term “therapeutically effective amount” as used herein shall mean that amount of a drug or pharmaceutical agent that will elicit the biological or medical response of a tissue, system, animal or human that is being sought by a researcher or clinician.

Accordingly, the present invention includes within its scope the use of a muscarinic M1 receptor ectopic activator, alone or in combination with other agents, for the subject indications in a warm-blooded animal. For the purposes of this disclosure, a warm-blooded animal is a member of the animal kingdom which includes but is not limited to mammals and birds. The preferred mammal for purposes of this invention is human.

The subject treated in the present methods is generally a mammal, preferably a human, male or female, in whom activation of muscarinic M1 receptor activity is desired. In the present invention, it is preferred that the subject mammal is a human. Although the present invention is applicable to both old and young people, in certain aspects such as cognition enhancement it would find greater application in elderly people.

The term “composition” as used herein is intended to encompass a product comprising the specified ingredients in the specified amounts, as well as any product which results, directly or indirectly, from combination of the specified ingredients in the specified amounts. Such term in relation to pharmaceutical composition is intended to encompass a product comprising the active ingredient(s), and the inert ingredient(s) that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present invention encompass any composition made by admixing a compound of the present invention and a pharmaceutically acceptable carrier. By “pharmaceutically acceptable” it is meant the carrier, diluent or excipient must be compatible with the other ingredients of the formulation and not deleterious to the recipient thereof.

The terms “administration of” and or “administering a” compound should be understood to mean providing a compound of the invention or a prodrug of a compound of the invention to the individual in need of treatment.

This particular application of muscarinic M1 receptor ectopic activators provides unexpected benefit relative to the administration of other agents for the subject indications. Muscarinic M1 receptor ectopic activators which are orally active also have the benefit being able to be administered orally, rather than just intravenously, intraperitoneally or subcutaneously. In an embodiment of the present invention, activation of the M1 muscarinic receptor selectively stimulates M1 receptors in a physiological way without the side effects associated with the blockade of M2, M3, M4 or M5 muscarinic receptors.

Although the specific mechanism underlying the present invention is not currently understood, the inventors postulate that cholinergic projections from the medial septum act on muscarinic acetylcholine receptors (mAChRs) in the basal forebrain and frontal cortex where they play a critical role in memory and attention mechanisms. Compounds that increase cholinergic and mAChR function are thus useful as therapeutic agents for treatment of patients with Alzheimer's disease (AD) and other cognitive disorders. The viability of this approach has been substantiated by the introduction of tacrine and other acetylcholinesterase (AChE) inhibitors into the clinic to improve cognitive performance and quality of life in AD patients. Unfortunately, ACHE inhibitors increase transmission at all cholinergic synapses and thereby induce adverse effects (AEs) that prevent administration of doses that could achieve their maximal possible efficacy. The most prominent AEs of these compounds are mediated by activation of peripheral mAChRs and include bradycardia, GI distress, excessive salivation, and sweating. It is proposed that M2, M3 and M5 mAChR subtypes mediate these unwanted AEs. In contrast, the M1 receptor subtype likely mediates the effects on cognition, attentional mechanisms, and sensory processing. Thus, mAChR agonists are useful therapeutic agents for treatment of schizophrenia and behavioral disturbances in AD patients. Also, mAChR agonists would possess utility as cognition-enhancing agents. These findings are particularly interesting in light of the prominent role of mAChRs, and especially M1, in regulating function of limbic, midbrain, and cortical regions that are disrupted in schizophrenia and other psychotic states. M1 plays a critical role in regulating dopaminergic function so that mAChR agonists inhibit dopamine (“DA”) release in nucleus accumbens and PFC, whereas DA release is increased by mAChR antagonists and in M1 knockout mice. In addition to the utility of M1 ectopic activators for providing efficacy in treating behavioral disturbances in AD patients and as antipsychotics for treatment of schizophrenia, M1 ectopic activators have potential for providing some improvement of cognitive function in AD patients. In addition, M1 ectopic activators may be useful in reducing intraocular pressure (IOP) without inducing the unacceptable adverse effects of non-selective mAChR agonists. M1 ectopic activators also have potential for use in treatment of neuropathic pain.

The present invention includes within its scope a pharmaceutical composition for the subject indications comprising, as an active ingredient, at least one muscarinic M1 receptor ectopic activator in association with a pharmaceutical carrier or diluent. Optionally, the active ingredient of the pharmaceutical compositions can comprise another agent in addition to at least one muscarinic M1 receptor ectopic activator to enhance efficacy or minimize side effects.

The present invention is further directed to a method for the manufacture of a medicament for the subject indications in humans comprising combining a compound that is a muscarinic M1 receptor ectopic activator with a pharmaceutical carrier or diluent.

It will be known to those skilled in the art that there are numerous compounds now being used for schizophrenic disorders, psychosis, enhancing cognition, and the like. Combinations of these therapeutic agents (some of which have also been mentioned herein) with a muscarinic M1 receptor ectopic activator will bring additional, complementary, and often synergistic properties to enhance the desirable properties of these various therapeutic agents. In these combinations, the muscarinic M1 receptor ectopic activator and the therapeutic agents may be independently present in dose ranges from one one-hundredth to one times the dose levels which are effective when these agents are used singly.

The muscarinic M1 receptor ectopic activator may be administered in combination with sedatives, hypnotics, anxiolytics, antipsychotics, antianxiety agents, minor tranquilizers, melatonin agonists and antagonists, melatonergic agents, benzodiazepines, barbiturates, 5HT-2 antagonists, and the like, or the muscarinic M1 receptor ectopic activator may be administered in conjunction with the use of physical methods such as electrical stimulation.

Suitable agents for use in combination with a muscarinic M1 receptor ectopic activator include typical antipsychotics and atypical antipsychotics, such as the phenothiazine, thioxanthene, heterocyclic dibenzazepine, butyrophenone, diphenylbutylpiperidine and indolone classes of antipsychotic agents. Suitable examples of phenothiazines include chlorpromazine, mesoridazine, thioridazine, acetophenazine, fluphenazine, perphenazine and trifluoperazine. Suitable examples of thioxanthenes include chlorprothixene and thiothixene. Suitable examples of dibenzazepines include clozapine and olanzapine. An example of a butyrophenone is haloperidol. An example of a diphenylbutylpiperidine is pimozide. An example of an indolone is molindolone. Other antipsychotic agents include loxapine, sulpiride and risperidone. It will be appreciated that the antipsychotic agents when used in combination with a muscarinic M1 receptor ectopic activator may be in the form of a pharmaceutically acceptable salt, for example, chlorpromazine hydrochloride, mesoridazine besylate, thioridazine hydrochloride, acetophenazine maleate, fluphenazine hydrochloride, fluphenazine enanthate, fluphenazine decanoate, trifluoperazine hydrochloride, thiothixene hydrochloride, haloperidol decanoate, loxapine succinate and molindone hydrochloride. Perphenazine, chlorprothixene, clozapine, olanzapine, haloperidol, pimozide and risperidone are commonly used in a non-salt form.

Other classes of antipsychotic agents suitable for use in combination with a muscarinic M1 receptor ectopic activator include dopamine receptor antagonists, especially D2, D3 and D4 dopamine receptor antagonists. An example of a D3 dopamine receptor antagonist is the compound PNU-99194A. An example of a D4 dopamine receptor antagonist is PNU-101387. Another class of antipsychotic agent for use in combination with a muscarinic M1 receptor ectopic activator is the 5-HT_{2A} receptor antagonists, examples of which include MDL100907 and fananserin. Also of use in combination with a NK-1 receptor antagonist are the serotonin dopamine antagonists (SDAs) which are believed to combine 5-HT_{2A} and dopamine receptor antagonist activity, examples of which include aripiprazole, olanzapine, quetiapine, risperidone and ziperasidone.

In a preferred aspect of the present invention, a muscarinic M1 receptor allosteric potentiator may be employed in combination with an acetylcholine esterase inhibitor.

In another preferred aspect of the present invention, a muscarinic M1 receptor ectopic agonist may be employed in combination with a 5HT_{2A} receptor antagonist. For example, the muscarinic M1 receptor ectopic activator may be given in combination with any such compounds and salts thereof, as well as admixtures and combinations thereof.

To illustrate these combinations, a muscarinic M1 receptor ectopic activator effective clinically at a given daily dose range may be effectively combined, at levels which are equal or less than the daily dose range, with such compounds at the indicated per day dose range. Typically, the individual daily dosages for these combinations may range from about one-fifth of the minimally recommended clinical dosages to the maximum recommended levels for the entities when they are given singly. It will be readily apparent to one skilled in the art that the muscarinic M1 receptor ectopic activator may be employed with other agents for the purposes of the present invention.

Naturally, these dose ranges may be adjusted on a unit basis as necessary to permit divided daily dosage and, as noted above, the dose will vary depending on the nature and severity of the disease, weight of patient, special diets and other factors.

These combinations may be formulated into pharmaceutical compositions as known in the art and as discussed below. A muscarinic M1 receptor ectopic activator may be administered alone or in combination by oral, parenteral (e.g., intramuscular, intraperitoneal, intravenous or subcutaneous injection, or implant), nasal, vaginal, rectal, sublingual, or topical routes of administration and can be formulated in dosage forms appropriate for each route of administration.

Solid dosage forms for oral administration include capsules, tablets, pills, powders and granules. In such solid dosage forms, the active compound is admixed with at least one inert pharmaceutically acceptable carrier such as sucrose, lactose, or starch. Such dosage forms can also comprise, as is normal practice, additional substances other than inert diluents, e.g., lubricating agents such as magnesium stearate. Illustrative of the adjuvants which may be incorporated in tablets, capsules and the like are the following: a binder such as gum tragacanth, acacia, corn starch or gelatin; an excipient such as microcrystalline cellulose; a disintegrating agent such as corn starch, pregelatinized starch, alginic acid and the like; a lubricant such as magnesium stearate; a sweetening agent such as sucrose, lactose or saccharin; a flavoring agent such as peppermint, oil of wintergreen or cherry. In the case of capsules, tablets and pills, the dosage forms may also comprise buffering agents. When the unit dosage form is a capsule, it may contain, in addition to materials of the above type, a liquid carrier such as a fatty oil. Various other materials may be present as coatings or to otherwise modify the physical form of the dosage unit. Tablets and pills can additionally be prepared with enteric coatings and tablets may be coated with shellac, sugar or both.

Liquid dosage forms for oral administration include pharmaceutically acceptable emulsions, solutions, suspensions, syrups, and elixirs, all of which may contain inert diluents commonly used in the art, such as water. Besides such inert diluents, compositions can also include adjuvants, such as wetting agents, emulsifying and suspending agents, and sweetening, flavoring, and perfuming agents. A syrup or elixir may contain the active compound, sucrose as a sweetening agent, methyl and propyl parabens as preservatives, a dye and a flavoring such as cherry or orange flavor.

Preparations according to this invention for parenteral administration include sterile aqueous or non-aqueous solutions, suspensions, or emulsions. Sterile compositions for injection may be formulated according to conventional pharmaceutical practice by dissolving or suspending the active substance in a vehicle such as water for injection, a naturally occurring vegetable oil like sesame oil, coconut oil, peanut oil, cottonseed oil, etc., or a synthetic fatty vehicle like ethyl oleate or the like. Buffers, preservatives, antioxidants and the like may be incorporated as required. Examples of non-aqueous solvents or vehicles are propylene glycol, polyethylene glycol, vegetable oils, such as olive oil and corn oil, gelatin, and injectable organic esters such as ethyl oleate. Such dosage forms may also contain adjuvants such as preserving, wetting, emulsifying, and dispersing agents. They may be sterilized by, for example, filtration through a bacteria-retaining filter, by incorporating sterilizing agents into the compositions, by irradiating the compositions, or by heating the compositions. They can also be manufactured in the form of sterile solid compositions which can be dissolved in sterile water, or some other sterile injectable medium immediately before use. Compositions for rectal or vaginal administration may be suppositories which may contain, in addition to the active substance, excipients such as cocoa butter or a suppository wax. Compositions for nasal or sublingual administration are also prepared with standard excipients well known in the art.

The dosage of active ingredient in the compositions of this invention may be varied, however, it is necessary that the amount of the active ingredient be such that a suitable dosage form is obtained. The active ingredient may be administered to patients (animals and human) in need of such treatment in dosages that will provide optimal pharmaceutical efficacy. The selected dosage depends upon the desired therapeutic effect, on the route of administration, and on the duration of the treatment. The dose will vary from patient to patient depending upon the nature and severity of disease, the patient's weight, special diets then being followed by a patient, concurrent medication, and other factors which those skilled in the art will recognize. Generally, dosage levels of between 0.0001 to 10 mg/kg. of body weight daily are administered to the patient, e.g., humans and elderly humans, to obtain effective antagonism of the muscarinic M1 receptor. The dosage range will generally be about 0.5 mg to 1.0 g. per patient per day which may be administered in single or multiple doses. Preferably, the dosage range will be about 0.5 mg to 500 mg per patient per day; more preferably about 0.5 mg to 200 mg per patient per day; and even more preferably about 5 mg to 50 mg per patient per day.

Pharmaceutical compositions of the present invention may be provided in a solid dosage formulation preferably comprising about 0.5 mg to 500 mg active ingredient, more preferably comprising about 1 mg to 250 mg active ingredient. The pharmaceutical composition is preferably provided in a solid dosage formulation comprising about 1 mg, 5 mg, 10 mg, 25 mg, 50 mg, 100 mg, 200 mg or 250 mg active ingredient.

While the invention has been described and illustrated with reference to certain particular embodiments thereof, those skilled in the art will appreciate that various adaptations, changes, modifications, substitutions, deletions, or additions of procedures and protocols may be made without departing from the spirit and scope of the invention. For example, effective dosages other than the particular dosages as set forth herein above may be applicable as a consequence of variations in the responsiveness of the mammal being treated for any of the indications with the compounds of the invention indicated above. Likewise, the specific pharmacological responses observed may vary according to and depending upon the particular active compounds selected or whether there are present pharmaceutical carriers, as well as the type of formulation and mode of administration employed, and such expected variations or differences in the results are contemplated in accordance with the objects and practices of the present invention. It is intended, therefore, that the invention be defined by the scope of the claims which follow and that such claims be interpreted as broadly as is reasonable.

1. A method for treating, controlling, ameliorating or reducing the risk of psychosis in a patient in need thereof that comprises administering to the patient a therapeutically effective amount of a muscarinic M1 receptor ectopic activator.

2. The method of claim 1, wherein the psychosis is a schizophrenic disorder.

3. The method of claim 1, wherein the patient is non-responsive to antipsychotic agents, or the patient is one for whom antipsychotic agents are contraindicated.

4. The method of claim 1, wherein the psychosis is associated with Alzheimer's disease.

5. The method of claim 1, wherein psychosis is associated with bipolar disorder.

6. The method of claim 2 wherein the schizophrenic disorder is selected from: paranoid, disorganized, catatonic, undifferentiated and residual schizophrenia; schizophreniform disorder; schizoaffective disorder; delusional disorder; brief psychotic disorder; shared psychotic disorder; substance-induced psychotic disorder; and psychotic disorder not otherwise specified.

7. The method of claim 1 wherein the muscarinic M1 receptor ectopic activator is a selective muscarinic M1 receptor ectopic activator.

8. The method of claim 1 wherein the muscarinic M1 receptor ectopic activator is a muscarinic M1 receptor allosteric potentiator.

9. The method of claim 1 wherein the muscarinic M1 receptor ectopic activator is a muscarinic M1 receptor ectopic agonist.

10. The method of claim 1 wherein the muscarinic M1 receptor ectopic activator possesses a selectivity for the muscarinic M1 receptor relative to each of the muscarinic M2, M3 and M5 receptors of at least 5 fold as measured by the ratio of EC₅₀ for the muscarinic M1 receptor to the EC₅₀ for each of the muscarinic M2, M3 and M5 receptors as evaluated by the Muscarinic FLIPR assay.

11. The method of claim 1 wherein the muscarinic M1 receptor ectopic activator possesses a selectivity for the muscarinic M1 receptor relative to a dopamine D2 receptor of at least 5 fold as measured by the ratio of EC₅₀ for the muscarinic M1 receptor as evaluated by the Muscarinic FLIPR assay to the EC₅₀ of the dopamine D2 receptor.

12. The method of claim 1 wherein the muscarinic M1 receptor ectopic activator possesses a selectivity for the muscarinic M1 receptor relative to all 5HT receptors of at least 5 fold as measured by the ratio of EC₅₀ for the muscarinic M1 receptor as evaluated by the Muscarinic FLIPR assay to the EC₅₀ of each of the 5HT receptors.

13. The method of claim 1 wherein the muscarinic M1 receptor ectopic activator acts at a different site than the orthosteric site of the muscarinic M1 receptor.

14. The method of claim 1 wherein the muscarinic M1 receptor ectopic activator possesses an EC₅₀ for binding to the muscarinic M1 receptor of 1 μM or less as evaluated by the Muscarinic FLIPR assay.

15. The method of claim 1 wherein the muscarinic M1 receptor ectopic activator is orally administered.

16. The method of claim 1 wherein the muscarinic M1 receptor ectopic activator is a non-peptidal muscarinic M1 receptor ectopic activator.

17. The method of claim 1 wherein the muscarinic M1 receptor ectopic activator is desmethylclozapine.

18. The method of claim 1 wherein the muscarinic M1 receptor ectopic activator is brucine.

19. A method for enhancing cognition in a patient in need thereof that comprises administering to the patient a therapeutically effective amount of a muscarinic M1 receptor ectopic activator.

20. A method for treating, controlling, ameliorating or reducing the risk of Alzheimer's disease in a patient in need thereof that comprises administering to the patient a therapeutically effective amount of a muscarinic M1 receptor ectopic activator.
